(19) United States
(12) Patent Application Publication
(10) Pub. No.: US 2002/0164266 A1
(43) Pub. Date: Nov. 7, 2002

(54) Microbicidal mixtures

(76) Inventors: Peter Wachtler, Krefeld (DE); Martin Kugler, Leichlingen (DE)

Correspondence Address: BAYER CORPORATION, PATENT DEPARTMENT, 100 BAYER ROAD, PITTSBURGH, PA, 15205, US

(21) Appl. No.: 10/096,557

(22) Filed: Mar. 13, 2002

(30) Foreign Application Priority Data
Mar. 15, 2001 (DE) 10112367.1

(51) Int. Cl. A61K 31/425; A61K 31/045

(52) U.S. Cl. 422/37; 514/372; 514/727

(57) ABSTRACT

The invention relates to synergistic mixtures of methyl-2H-isothiazol-3-one and 2-bromo-2-nitropropanediol for controlling microorganisms in industrial materials.

### BACKGROUND

The present invention relates to novel microbicidal mixtures, to processes for their preparation, to novel microbicidal compositions based on these mixtures and to the use of these mixtures and compositions for protecting industrial materials against attack by microorganisms.

It has been known for a long time that the active compound class of the 4-isothiazolin-3-ones can be used for preparing microbicidal formulations. Microbicidal mixtures comprising compounds from this class of active compounds are known, too, for example the 3:1 mixture of 5-chloro-2-methyl-2H-isothiazol-3-one (CMIT) and 2-methyl-2 H-isothiazol-3-one (MIT), which is frequently used commercially. However, this mixture known from the prior art can be improved upon, in particular with respect to its toxicological acceptability. Namely, in connection with certain applications, a certain allergizing action is prescribed to the component CMIT. However, the component MIT applied on its own does not achieve the required broad and rapid microbiological action.

It was an object of the present invention to provide a novel microbicidal mixture which does not have the disadvantages outlined above.

We have found novel mixtures of 2-methyl-2H-isothiazol-3-one (MIT) and 2-bromo-2-nitropropanediol (bronopol=BNPD) which have good toxicological acceptability and, surprisingly, synergistic action.

The active compound bronopol is likewise known and used in industrial preservation, in industrial process control and in the preservation of cosmetic products. The activity spectrum of bronopol includes both gram-positive and gram-negative bacteria and, in higher concentrations, also fungi and yeasts.

### SUMMARY

The invention relates to a mixture comprising an antimicrobially effective amount of 2-methyl-2H-isothiazol-3-one and 2-bromo-2-nitro-1,3-propanediol.

In one embodiment, the invention relates to a microbicidal composition comprising a mixture containing an antimicrobially effective amount of 2-methyl-2H-isothiazol-3-one and 2-bromo-2-nitro-1,3-propanediol, extenders and, optionally, surfactants.

In another embodiment, the invention relates to a method for protecting an industrial material against attack from a microorganism comprising mixing or treating a material with (i) a mixture comprising an antimicrobially effective amount of 2-methyl-2H-isothiazol-3-one and 2-bromo-2-nitro-1,3-propanediol or (ii) a microbicidal composition comprising a mixture containing an antimicrobially effective amount of 2-methyl-2H-isothiazol-3-one and 2-bromo-2-nitro-1,3-propanediol, extenders and, optionally, surfactants.

In another embodiment, the invention relates to a process for preparing a microbicidal composition comprising a mixture containing an antimicrobially effective amount of 2-methyl-2H-isothiazol-3-one and 2-bromo-2-nitro-1,3-propanediol, extenders and, optionally, surfactants,

the process comprising mixing (i) a mixture comprising an antimicrobially effective amount of 2-methyl-2H-isothiazol-3-one and 2-bromo-2-nitro-1,3-propanediol with an extender and, optionally, a surfactant

These and other features, aspects, and advantages of the present invention will become better understood with reference to the following description and appended claims.

### DESCRIPTION

Accordingly, the present invention relates to mixtures comprising an antimicrobially effective amount of 2-methyl-2H-isothiazol-3-one (MIT) and 2-bromo-2-nitropropanediol (BNPD).

The mixture according to the invention is characterized by a synergistic mode of action. This means that the amounts of active compounds which have to be used for protecting industrial products can be reduced, compared to the required concentrations in the case of the respective individual compounds, which results in a more economical use, is advantageous with respect to the application and contributes to an increased preservation quality. The mixtures according to the invention can be produced in a known manner by mixing the individual components.

The mixtures according to the invention are highly active against microorganisms. They can be used in the protection of materials for protecting industrial materials, in particular for protecting aqueous industrial liquids and aqueous products, against attack by undesirable microorganisms such as bacteria and mould and also yeasts and slime organisms. The following microorganisms may be mentioned by way of example, without imposing any limitations:

Alternaria, such as Alternaria tenuis, Aspergillus, such as Aspergillus niger, Chaetomium, such as Chaetomium globosum, Fusarium, such as Fusarium solani, Lentinus, such as Lentinus tigrinus, Penicillium, such as Penicillium glaucum;

Alcaligenes, such as Alcaligenes faecalis, Bacillus, such as Bacillus subtilis, Escherichia, such as Escherichia coli, Pseudomonas, such as Pseudomonas aeruginosa or Pseudomonas fluorescens, Staphylococcus, such as Staphylococcus aureus; Candida, such as Candida albicans, Geotrichum, such as Geotrichum candidum.

The mixtures according to the invention are preferably suitable for protecting industrial water-containing liquids and water-containing industrial products susceptible to attack by microorganisms.

By way of example, but not by way of limitation, the following water-containing industrial liquids and products may be mentioned:

paints, colors, plasters and other coating materials

starch solutions, dispersions or slurries or other starch-based products, such as, for example, thickeners used in printing

slurries of other raw materials such as color pigments (for example iron oxide pigments, carbon black pigments, titanium dioxide pigments) or slurries of fillers or spreadable pigments such as kaolin or calcium carbonate

concrete additives, for example those based on molasses or lignosulfonates

glues and adhesives based on known raw materials of animal, vegetable or synthetic origin

polymer dispersions based, for example, on polyacrylate, polystyrene-acrylate, styrene-butadiene etc.

detergents and cleaners for industrial and domestic use

mineral oils or mineral oil products (such as, for example, diesel fuels)

auxiliaries for the leather, textile or photochemical industry

precursors and intermediates of the chemical industry, for example in the production and storage of dyestuffs

solventborne or waterborne inks

wax and clay emulsions.

The mixtures according to the invention may additionally comprise one or more further biocidally active compounds. Mixing partners which may be mentioned by way of example are the following compounds:

benzyl alcohol

formaldehyde and formaldehyde depot substances

benzyl hemiformal

N-methylolurea

dimethylolurea

ethylene glycol hemiformal

ethylene glycol bis-hemiformal

1,2-dibromo-2,4-dicyanobutane

BIT (benzisothiazolinone)

Tektamer 38 (1,2-dibromo-2,4-dicyano-butane)

2-n-octylisothiazolin-3-one

iodopropargyl butylcarbamate.

The quantities of the components 2-methyl-2H-isothiazol-3-one (MIT) and bronopol (BNPD) contained in the mixtures according to the invention can be varied within a wide range. To achieve a broad antimicrobial action, the ratio of methyl-2H-isothiazol-3-one (MIT) to bronopol (BNPD) is usually a weight ratio of from about 100:1 to about 1:50, preferably from about 15:1 to about 1:10, particularly preferably from about 5:1 to about 1:5.

The mixtures according to the invention can be metered into the water-containing industrial products or liquids, depending on their respective physical and/or chemical properties, either separately in the form of the individual active compounds, where it is possible, depending on the present preservation problem, to adjust the concentration ratio individually, or else it is possible to use the mixture of active compounds directly, after conversion into a customary formulation, such as, for example, a solution, emulsion, suspension, foam, paste, aerosol, or as a microencapsulation in polymeric substances.

These formulations can be prepared in a manner known per se, for example by mixing the active compounds with extenders, that is liquid solvents, liquefied gases under pressure and/or solid carriers, optionally with the use of surfactants, that is emulsifiers and/or dispersants and/or foam formers. If the extender used is water, it is also possible to use, for example, organic solvents as auxiliary solvents. Essentially, suitable liquid solvents include: alcohols, such as butanol or glycol and their ethers and esters, ketones, such as acetone, methyl ethyl ketone, methyl isobutyl ketone or cyclohexanone, strongly polar solvents, such as dimethylformamide or dimethyl sulfoxide, or else water; liquefied gaseous extenders or carriers are to be understood as meaning liquids which are gaseous at ambient temperature and under atmospheric pressure, for example aerosol propellants such as halogenated hydrocarbons and also butane, propane, nitrogen and carbon dioxide; suitable solid carriers are: for example ground natural minerals, such as kaolins, clays, talc, chalk, quartz, attapulgite, montmorillonite or diatomaceous earth, and ground synthetic minerals such as finely divided silica, aluminium oxide and silicates; suitable solid carriers for granules are: for example crushed and fractionated natural rocks such as calcite, marble, pumice, sepiolite and dolomite, and also synthetic granules of inorganic and organic meals, and granules of organic material such as sawdust, coconut shells, maize cobs and tobacco stalks; suitable emulsifiers and/or foam formers are: for example nonionic and anionic emulsifiers, such as polyoxyethylene fatty acid esters, polyoxyethylene fatty alcohol ethers, for example alkylaryl polyglycol ethers, alkylsulfonates, alkyl sulfates, arylsulfonates, and also protein hydrolysates; suitable dispersants are: for example lignosulfite waste liquors and methylcellulose.

Tackifiers such as carboxymethylcellulose and natural and synthetic polymers in the form of powders, granules or lattices, such as gum arabic, polyvinyl alcohol and polyvinyl acetate, and also natural phospholipids such as cephalins and lecithins and synthetic phospholipids can be used in the formulations. Other possible additives are mineral and vegetable oils.

The present invention also provides microbicidal compositions comprising a mixture of 2-methyl-2H-isothiazol-3-one and 2-bromo-2-nitropropanediol and also extenders and, if appropriate, surfactants.

The microbicidal compositions according to the invention comprise the active compounds methyl-2H-isothiazol-3-one (MIT) and bronopol (BNPD) in a concentration of (based on the sum of the individual active compounds) from about 0.5 to about 50% by weight, preferably from about 1.0 to about 30% by weight, in particular from about 5.0 to about 20% by weight, and also from about 5 to about 30% by weight of extenders and, if appropriate, from about 0.5 to about 5.0% by weight of surfactants.

The use concentrations of the mixtures to be used according to the invention depend on the nature and the occurrence of the microorganisms to be controlled and on the composition of the material to be protected. The optimum amount for use can be determined by test series. In general, the use concentrations are in the range from about 0.01 to about 2% by weight, preferably from about 0.05 to about 1.0% by weight, of the mixture according to the invention, based on the material to be protected.

The mixtures according to the invention have synergistic activity. The observed synergism can be determined by the following mathematical equation (see F. C. Kull, P. C. Elisman, H. D. Sylwestrowicz and P. K. Mayer, Appl. Microbiol. 9, 538 (1961):

$$Synergistic Index ( SI ) = \frac{{Q}_{a}}{{Q}_{A}} + \frac{{Q}_{b}}{{Q}_{B}}$$

where

Qₐ=the amount of component A in the active compound mixture which achieves the desired effect, i.e. no microbial growth.

Q_{A}=the amount of component A which, when used on its own, suppresses the growth of the microorganisms.

Q_{b}=the amount of component B in the active compound mixture which suppresses the growth of the micoorganisms.

Q_{B}=the amount of component B which, when used on its own, suppresses the growth of the microorganisms.

A synergistic index of SI<1 indicates a synergistic effect for the mixture in question. The synergistically enhanced activity of the mixtures according to the invention is documented by way of example, without imposing any limitation, by the calculations below, in which the compounds indicated in Tables 1, 2, and 3 below were mixed. The results are shown in Tables 1, 2, and 3. All parts and percentages are by weight unless otherwise indicated.

### EXAMPLES

### Example 1

Synergism (BNPD/MIT)

Test pathogen: Bacillus subtilis

In brackets=mixing ratios of the pure active compounds

TABLE 1
$Synergistic Index ( SI ) = \frac{{Q}_{a}}{{Q}_{A}} + \frac{{Q}_{b}}{{Q}_{B}}$

| Compound/ | MIC values for Bacillus subtilis | |
|---|---|---|
| Mixture | (in ppm) | SI |
| BNPD (Q_{A}) | 10 | — |
| MIT (Q_{B}) | 25 | — |
| | Qₐ/Q_{b} | |
| BNPD/MIT (50:1) | 5/0.1 | 0.50 |
| BNPD/MIT (25:1) | 3.75/0.15 | 0.38 |
| BNPD/MIT | 5/0.4 | 0.52 |
| (12.5:1) | | |
| BNPD/MIT (10:1) | 2.5/0.25 | 0.28 |
| BNPD/MIT (7.5:1) | 2.5/0.33 | 0.25 |
| BNPD/MIT (5:1) | 1.5/0.3 | 0.16 |
| BNPD/MIT (2.5:1) | 1.5/0.6 | 0.16 |
| BNPD/MIT (1:1) | 1.25/1.25 | 0.17 |
| BNPD/MIT (1:2.5) | 0.8/2 | 0.16 |
| BNPD/MIT (1:5) | 0.4/2 | 0.12 |
| BNPD/MIT (1:7.5) | 0.5/3.75 | 0.20 |
| BNPD/MIT (1:10) | 0.5/5 | 0.25 |
| BNPD/MIT (1:25) | 0.5/12.5 | 0.55 |

The combinations according to the invention have pronounced synergistic activity.

### Example 2

Synergism (BNPD/MIT)

Test pathogen: Pseudomonas fluorescens

In brackets=mixing ratios of the pure active compounds

TABLE 2
$Synergistic Index ( SI ) = \frac{{Q}_{a}}{{Q}_{A}} + \frac{{Q}_{b}}{{Q}_{B}}$

| | MIC values for Pseudomonas | |
|---|---|---|
| Compound/ | fluorescens | |
| Mixture | (in ppm) | SI |
| BNPD (Q_{A}) | 10 | — |
| MIT (Q_{B}) | 10 | — |
| | Qₐ/Q_{b} (in ppm) | |
| BNPD/MIT (50:1) | 5/0.1 | 0.51 |
| BNPD/MIT (25:1) | 5/0.2 | 0.52 |
| BNPD/MIT (12.5:1) | 5/0.4 | 0.54 |
| BNPD/MIT (10:1) | 2.5/0.25 | 0.28 |
| BNPD/MIT (7.5:1) | 2.5/0.33 | 0.25 |
| BNPD/MIT (5:1) | 2/0.4 | 0.24 |
| BNPD/MIT (2.5:1) | 2/0.8 | 0.28 |
| BNPD/MIT (1:1) | 1.25/1.25 | 0.25 |
| BNPD/MIT (1:2.5) | 0.8/2 | 0.28 |
| BNPD/MIT (1:5) | 0.4/2 | 0.24 |
| BNPD/MIT (1:7.5) | 0.33/2.5 | 0.28 |
| BNPD/MIT (1:10) | 0.25/2.5 | 0.27 |
| BNPD/MIT (1:25) | 0.1/2.5 | 0.26 |

The mixtures according to the invention show a clear synergistic activity.

### Example 3

Synergism (BNPD/MIT)

Test pathogen: Pseudomonas aeruginosa

In brackets=mixing ratios of the pure active compounds

TABLE 3
$Synergistic Index ( SI ) = \frac{{Q}_{a}}{{Q}_{A}} + \frac{{Q}_{b}}{{Q}_{B}}$

| | MIC values for Pseudomonas | |
|---|---|---|
| Compound/ | aeruginosa | |
| Mixture | (in ppm) | SI |
| BNPD (Q_{A}) | 10 | — |
| MIT (Q_{B}) | 10 | — |
| | Qₐ/Q_{b} (in ppm) | |
| BNPD/MIT (50:1) | 12.5/0.25 | >1 |
| BNPD/MIT (25:1) | 12.5/0.5 | >1 |
| BNPD/MIT | 12.5/1 | >1 |
| (12.5:1) | | |
| BNPD/MIT (10:1) | 5/0.5 | 0.55 |
| BNPD/MIT (7.5:1) | 5/0.66 | 0.57 |
| BNPD/MIT (5:1) | 4/0.8 | 0.48 |
| BNPD/MIT (2.5:1) | 4/1.6 | 0.56 |
| BNPD/MIT (1:1) | 1.8/1.8 | 0.36 |
| BNPD/MIT (1:2.5) | 0.8/2 | 0.28 |
| BNPD/MIT (1:5) | 0.4/2 | 0.24 |
| BNPD/MIT (1:7.5) | 0.33/2.5 | 0.28 |
| BNPD/MIT (1:10) | 0.25/2.5 | 0.28 |
| BNPD/MIT (1:25) | 0.1/2.5 | 0.26 |

In the case of Pseudomonas aeruginosa, the combinations according to the invention show a clear synergistic activity if the excess of BNPD, based on MIT, is not greater than 10:1.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. A mixture comprising an antimicrobially effective amount of 2-methyl-2H-isothiazol-3-one and 2-bromo-2-nitro-1,3-propanediol.

2. The mixture according to claim 1, wherein the weight ratio of the methyl-2H-isothiazol-3-one to the 2-bromo-2-nitropropanediol is from about 100:1 to about 1:50.

3. A microbicidal composition comprising a mixture containing an antimicrobially effective amount of 2-methyl-2H-isothiazol-3-one and 2-bromo-2-nitro-1,3-propanediol, extenders and, optionally, surfactants.

4. A method for protecting an industrial material against attack from a microorganism comprising mixing or treating a material with (i) a mixture comprising (a) an antimicrobially effective amount of 2-methyl-2H-isothiazol-3-one and (b) 2-bromo-2-nitro-1,3-propanediol or (ii) a microbicidal composition comprising (a) a mixture containing an antimicrobially effective amount of 2-methyl-2H-isothiazol-3-one and 2-bromo-2-nitro-1,3-propanediol, (b) and extender and, (c) optionally, a surfactant.

5. The method according to claim 4, wherein the industrial material is a water-containing industrial liquid or a water-containing industrial product.

6. A process for preparing a microbicidal composition comprising a mixture containing (a) an antimicrobially effective amount of 2-methyl-2H-isothiazol-3-one and 2-bromo-2-nitro-1,3-propanediol, (b) an extender and, (c) optionally, a surfactant,
the process comprising mixing (i) a mixture comprising an antimicrobially effective amount of 2-methyl-2H-isothiazol-3-one and 2-bromo-2-nitro-1 ,3-propanediol with an extender and, optionally, a surfactant.

7. The process according to claim 6, wherein the mixture further comprises an extender and, optionally, a surfactant, and wherein the methyl-2H-isothiazol-3-one and the 2-bromo-2-nitropropanediol have a weight ratio ranging from about 100:1 to about 1:50.

8. The process according to claim 6, wherein the process further comprises mixing the mixture with a solvent or solvent mixtures.
